(19) United States
(12) Patent Application Publication
(10) Pub. No.: US 2020/0330735 A1
(43) Pub. Date: Oct. 22, 2020

(54) BALLOON CATHETER

(71) Applicant: ASAHI INTECC CO., LTD., Seto-shi (JP)

(72) Inventors: Akihiro MIKI, Seto-shi (JP); Yutaka ERIKAWA, Seto-shi (JP)

(73) Assignee: ASAHI INTECC CO., LTD., Seto-shi (JP)

(21) Appl. No.: 16/916,213

(22) Filed: Jun. 30, 2020

Related U.S. Application Data
(63) Continuation of application No. PCT/JP2018/011780, filed on Mar. 23, 2018.

(51) Int. Cl. A61M 25/10

(52) U.S. Cl. CPC A61M 25/10; A61M 2025/0004; A61M 2025/1079

(57) ABSTRACT

A balloon catheter is provided with a balloon; an outer shaft that is coupled to a proximal end of the balloon with a first coupling part; an inner shaft that is inserted into the outer shaft and that is coupled to a distal end of the balloon with a second coupling part; and a proximal end-side marker disposed on the inner shaft. The ends of the proximal end-side marker are disposed astride at least one of a distal end coupling position and a proximal end coupling position of the first coupling part.

### CROSS REFERENCE TO RELATED APPLICATION

This is a Continuation of PCT/JP2018/011780 filed Mar. 23, 2018. The disclosure of the prior application is hereby incorporated by reference herein in its entirety.

### BACKGROUND

The disclosed embodiments relate to a balloon catheter used in the medical field.

Conventionally, a therapeutic balloon catheter that restores the flow of blood or digestive fluid by expanding a stenosis formed inside a body lumen such as a blood vessel of a patient, and a fixing balloon catheter that facilitates operation of a medical device such as a guide wire inserted therein by expanding and fixing a balloon to a blood vessel wall or a digestive organ wall, are known.

A balloon catheter is typically provided with a balloon that can be expanded and contracted, an outer shaft that is coupled to the proximal end of the balloon, and an inner shaft that is inserted into the balloon and the outer shaft and that is coupled to the distal end of the balloon. Here, the inner shaft is used for inserting a guide wire, and a lumen provided between the outer shaft and the inner shaft is used to charge and discharge a liquid (a liquid such as a physiological saline or a contrast agent) for expanding and contracting the balloon.

Furthermore, a radiopaque marker is provided inside the balloon of the balloon catheter so that the position of the balloon can be confirmed during a procedure.

For example, Japanese Patent No. 6,103,721 describes a balloon catheter including a balloon, an outer tube shaft (hereinafter, referred to as an outer shaft) that is coupled to the proximal end of the balloon, an inner tube shaft (hereinafter, referred to as an inner shaft) that is inserted into the outer shaft and that is coupled to the distal end of the balloon, and a distal end-side contrast marker and a proximal end-side contrast marker which are formed on the inner shaft (see FIG. 2 and the like).

In the balloon catheter described in Japanese Patent No. 6,103,721, the distal end of the balloon is coupled to the inner shaft, and the proximal end of the balloon is coupled to the outer shaft.

However, when the balloon catheter is inserted into a body lumen of a patient and the balloon catheter becomes curved, stress concentrates on the coupling part for coupling the balloon and the outer shaft and the coupling part for coupling the balloon and the inner shaft, which causes a problem in that peeling occurs more easily at the coupling parts.

Furthermore, in the balloon catheter described in Japanese Patent No. 6,103,721, because the proximal end-side contrast marker is disposed within a coupling width of the coupling part for coupling the balloon and the outer shaft, stress is further concentrated on the coupling part for coupling the balloon and the outer shaft, which causes a problem in that peeling occurs even more easily at the coupling parts.

### SUMMARY

The disclosed embodiments have been devised in response to the above problems associated with the conventional techniques, and an object of the disclosed embodiments is to provide a balloon catheter that can prevent peeling between a balloon and an outer shaft or between the balloon and an inner shaft, and can prevent kinking near the end section of the balloon even when the balloon catheter is curved.

In order to address the above problems, a balloon catheter is provided with a balloon, an outer shaft that is coupled to a proximal end of the balloon with a first coupling part, an inner shaft that is inserted into the outer shaft and that is coupled to a distal end of the balloon with a second coupling part, and a marker disposed on the inner shaft, in which both ends of the marker are disposed astride at least one of a distal end and a proximal end of the first coupling part, or at least one of a distal end and a proximal end of the second coupling part in a longitudinal direction of the inner shaft, and therefore, peeling can be prevented between the balloon and the outer shaft or between the balloon and the inner shaft, and kinking can be prevented near the end section of the balloon even when the balloon catheter is curved.

It is possible that both ends of the marker are disposed astride the distal end and the proximal end of the first coupling part, or the distal end and the proximal end of the second coupling part in a longitudinal direction of the inner shaft, and therefore, peeling can be further prevented between the balloon and the outer shaft or between the balloon and the inner shaft, and kinking can be further prevented near the end section of the balloon even when the balloon catheter is curved.

The distal end of the outer shaft may extend beyond the first coupling part on the distal end side, and therefore, in addition to the effects described above, the gradual change in rigidity in the longitudinal direction near the proximal end section of the balloon can be made smoother, and further, peeling can be further prevented between the balloon and the outer shaft, and kinking can be further prevented near the end section of the balloon even when the balloon catheter is curved.

Additionally, a distal end of the marker, when positioned at the first coupling part, may extend beyond the distal end of the outer shaft on the distal end side, and therefore, in addition to the effects described above, peeling can be further prevented between the balloon and the outer shaft, and kinking can be further prevented near the end section of the balloon even when the balloon catheter is curved, and further, the balloon can be deflated more safely by preventing the necking phenomenon from occurring at the proximal end section of the balloon.

It is also possible that the marker is disposed at the first coupling part, and no marker is disposed at the second coupling part, and therefore, in addition to the effects described above, the distal end section of the balloon catheter can be made even thinner and softer.

### BRIEF DESCRIPTION OF DRAWINGS

FIG. 1 is an overall view of a balloon catheter according to the disclosed embodiments.

FIG. 2 is a longitudinal sectional view near the distal end section of the balloon catheter shown in FIG. 1, which is an enlarged view of section A in FIG. 1.

FIG. 3 is a longitudinal sectional view near the distal end section of a balloon catheter according to the disclosed embodiments.

FIG. 4 is a longitudinal sectional view near the distal end section of a balloon catheter according to the disclosed embodiments.

FIG. 5 is a longitudinal sectional view near the distal end section of a balloon catheter according to the disclosed embodiments.

FIG. 6 is a longitudinal sectional view near the distal end section of a balloon catheter according to the disclosed embodiments.

FIG. 7 is a longitudinal sectional view near the distal end section of a balloon catheter according to the disclosed embodiments.

### DETAILED DESCRIPTION OF EMBODIMENTS

Hereinafter, embodiments of the present invention will be described with reference to the drawings. The drawings are exaggerated for ease of understanding, and the dimensions may be different from the actual dimensions.

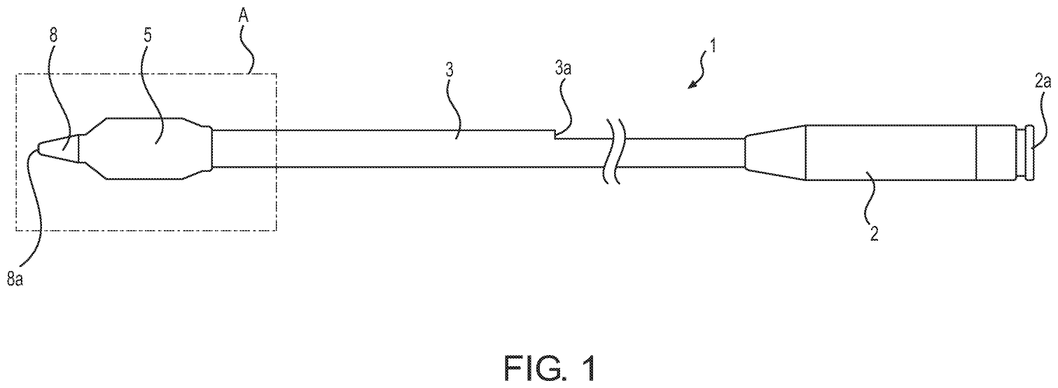

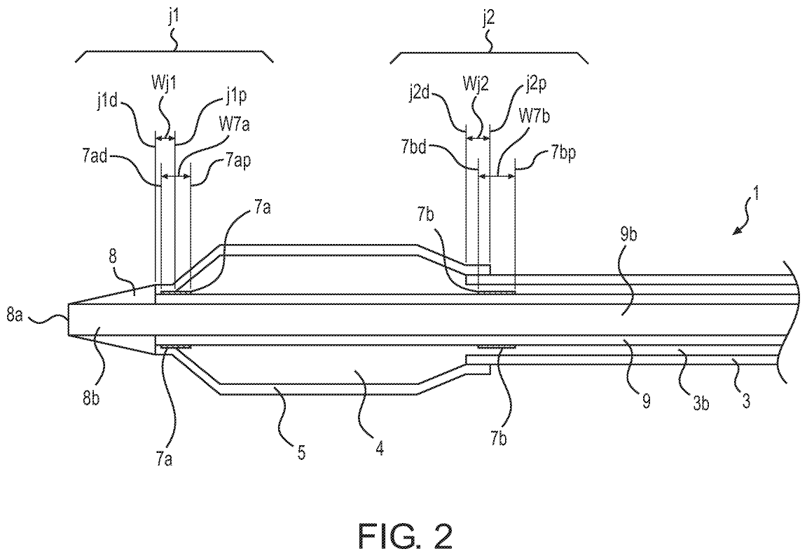

FIG. 1 is an overall view of a balloon catheter of the disclosed embodiments, and FIG. 2 is a longitudinal sectional view near the distal end section of the balloon catheter, which is an enlarged view of section A in FIG. 1.

As shown in FIG. 1, a balloon catheter 1 includes a long outer shaft 3, a balloon 5 provided on the distal end of the outer shaft 3, a distal tip 8 provided on the distal end of the balloon 5, an inner shaft 9 that is inserted into the outer shaft 3 and the balloon 5 and that is coupled to the distal end of the balloon 5 (see FIG. 2), and a connector 2 provided on the proximal end of the outer shaft 3.

The outer shaft 3 is a long and flexible tubular member, and as shown in FIG. 2, the distal end is coupled to the proximal end of the balloon 5, and the proximal end is coupled to the distal end of the connector 2. The outer shaft 3 has an inner shaft 9 inserted therein, and has a lumen 3b between the outer shaft 3 and the inner shaft 9 through which the internal space 4 of the balloon 5 communicates with a lumen inside the connector 2 (not illustrated).

Furthermore, the outer shaft 3 is provided with a guide wire port 3a along the longitudinal direction, which serves as an opening for inserting a guide wire, and the guide wire port 3a is coupled to the proximal end of the inner shaft 9.

The material forming the outer shaft 3 is a flexible material, and examples include polyolefins, such as polyethylene, polypropylene, ethylene-propylene copolymer, and ethylene-vinyl acetate copolymer, thermoplastic resins such as soft polyvinyl chloride, various rubbers such as silicone rubber and latex rubber, various elastomers such as polyurethane elastomers, polyamide elastomers, and polyester elastomers, and crystalline plastics such as polyamide, crystalline polyethylene, and crystalline polypropylene, and further, any material having biocompatibility can be used without particular limitation.

The balloon 5 is configured to expand and contract in response to increases and decreases in the internal volume caused by changes in the pressure inside the balloon 5. Specifically, the balloon 5 is constituted by a body portion that expands into a cylindrical shape, a distal end-side cone part which is provided such that the outer diameter gradually decreases from the body portion toward the distal end, and a proximal end-side cone part which is provided such that the outer diameter gradually decreases from the body portion toward the proximal end, and an internal space 4 is formed inside the balloon 5 when it expands.

As shown in FIG. 2, the balloon 5 has the distal end coupled to the distal end of the inner shaft 9, and the proximal end coupled to the distal end of the outer shaft 3. Therefore, a liquid such as a physiological saline which is charged from the opening 2a of the connector 2 described below passes through the inside of a lumen (not illustrated) of the connector 2 and the lumen 3b, and is stored in the internal space 4 inside the balloon 5, and the balloon 5 expands and contracts according to the amount of stored liquid.

The material forming the balloon 5 is preferably a material having some softness, and examples include polyolefins such as polyethylene, polypropylene, and ethylene-propylene copolymer, polyesters such as polyethylene terephthalate, thermoplastic resins such as polyvinyl chloride, ethylene-vinyl acetate copolymer, crosslinked ethylene-vinyl acetate copolymer, and polyurethane, polyamide, polyamide elastomers, silicone rubber, and latex rubber.

Furthermore, the balloon 5 may be formed having a single layer structure or having a laminated structure with two or more layers using the above materials. Moreover, the surface of the balloon 5 may be coated with a substance having an antithrombotic property.

The distal tip 8 is connected to the distal end of the balloon 5 and the distal end of the inner shaft 9, is provided with a lumen 8b which communicates with the lumen 9b of the inner shaft 9, and an opening 8a that constitutes the distal end of the lumen 8b, and has a tapered shape in which the outer diameter gradually decreases toward the distal end.

The material forming the distal tip 8 is preferably a material having some flexibility, and examples include polyurethane elastomers, polyolefins such as polyethylene, polypropylene, ethylene-propylene copolymer, and ethylene-vinyl acetate copolymer, thermoplastic resins such as soft polyvinyl chloride, polyamide, polyamide elastomers, and polyurethane, silicone rubber, and latex rubber, and the thermoplastic resins above are particularly preferable.

The inner shaft 9 is a long and flexible tubular member which is inserted inside the outer shaft 3 and the balloon 5. The distal end of the inner shaft 9 is coupled to the distal end of the balloon 5, and the proximal end of the inner shaft 9 is coupled to the guide wire port 3a of the outer shaft 3 and is provided with a lumen 9b therein.

The same material as the material forming the outer shaft 3 described above can be used as the material forming the inner shaft 9. The material is a flexible material, and examples include polyolefins, such as polyethylene, polypropylene, ethylene-propylene copolymer, and ethylene-vinyl acetate copolymer, thermoplastic resins such as soft polyvinyl chloride, various rubbers such as silicone rubber and latex rubber, various elastomers such as polyurethane elastomers, polyamide elastomers, and polyester elastomers, and crystalline plastics such as polyamide, crystalline polyethylene, and crystalline polypropylene, and any material having biocompatibility can be used without particular limitation.

Furthermore, the inner shaft 9 is provided with two radiopaque markers, namely a distal end-side marker 7a and a proximal end-side marker 7b, which enable the position of the balloon 5 to be confirmed during a procedure.

Next, the relationship between (i) the coupling position between the balloon 5 and the inner shaft 9, and (ii) the position of the distal end-side marker 7a will be described.

First, as shown in FIG. 2, the coupling position of the coupling part j1 between the balloon 5 and the inner shaft 9 (which corresponds to a “second coupling part”) has a distal end coupling position j1d at the distal end, and a proximal end coupling position j1p at the proximal end, which is at the position of a coupling width Wj1 on the proximal end side from the distal end coupling position j1d. In other words, the coupling width Wj1 is the distance between a distal end and a proximal end of the coupling part j1 in the longitudinal direction of the inner shaft 9 (or the balloon catheter 1).

In contrast, the distal end position lad of the distal end-side marker 7a is positioned on the proximal end side of the distal end coupling position j1d of the coupling part j1 in the longitudinal direction, and the proximal end position lap of the distal end-side marker 7a is positioned at a width W7a on the proximal end side from the distal end position lad, and is positioned on the proximal end side of the proximal end coupling position j1p of the coupling part j1 in the longitudinal direction.

That is to say, the ends of the distal end-side marker 7a are disposed astride (one end on either side of) the proximal end coupling position j1p of the coupling part j1 between the balloon 5 and the inner shaft 9 in the longitudinal direction, such that the proximal end coupling position j1p of the coupling part j1 is positioned between the ends of the distal end-side marker 7a in the longitudinal direction.

Therefore, because the ends of the distal end-side marker 7a are disposed astride the proximal end coupling position j1p of the coupling part j1 between the balloon 5 and the inner shaft 9 in the longitudinal direction, peeling can be prevented at the proximal end coupling position j1p between the balloon 5 and the inner shaft 9, and kinking can be prevented near the end section of the balloon 5 even when the balloon catheter 1 is curved.

Next, the relationship between (i) the coupling position between the balloon 5 and the outer shaft 3, and (ii) the position of the proximal end-side marker 7b will be described.

As shown in FIG. 2, the coupling position of the coupling part j2 between the balloon 5 and the outer shaft 3 (which corresponds to a “first coupling part”) has a distal end coupling position j2d at the distal end, and a proximal end coupling position j2p at the position of a coupling width Wj2 on the proximal end side from the distal end coupling position j2d.

In contrast, the distal end position 7bd of the proximal end-side marker 7b is positioned on the proximal end side of the distal end coupling position j2d of the coupling part j2 in the longitudinal direction, and the proximal end position 7bp is at the position of a width W7b on the proximal end side from the distal end position 7bd, and is positioned on the proximal end side of the proximal end coupling position j2p of the coupling part j2 in the longitudinal direction.

That is to say, the ends of the proximal end-side marker 7b are disposed astride the proximal end coupling position j2p of the coupling part j2 between the balloon 5 and the outer shaft 3 in the longitudinal direction.

Therefore, because the ends of the proximal end-side marker 7b are disposed astride the proximal end coupling position j2p of the coupling part j2 between the balloon 5 and the outer shaft 3 in the longitudinal direction, peeling can be prevented at the proximal end coupling position j2p between the balloon 5 and the outer shaft 3, and kinking can be prevented near the end section of the balloon 5 even when the balloon catheter 1 is curved.

Examples of the material that can be used to form the distal end-side marker 7a and the proximal end-side marker 7b include radiopaque metals or metallic compounds such as tungsten, gold, platinum, indium, platinum-iridium alloy, and tantalum, and radiopaque resins formed by kneading a radiopaque material such as tungsten, bismuth trioxide, or barium sulfate with a resin material.

The connector 2 is coupled to the proximal end of the outer shaft 3, and includes a lumen (not illustrated) that communicates with the lumen 3b of the outer shaft 3, and an opening 2a on the proximal end of the lumen, such that a liquid such as a physiological saline can be injected from a syringe connected to the opening 2a.

Examples of the material that can be used to form the connector 2 include thermoplastic resins such as polycarbonate, polyamide, polysulfone, polyarylate, and methacrylate-styrene copolymer.

The balloon catheter 1 is provided with a balloon 5, an outer shaft 3 that is coupled to a proximal end of the balloon 5 with a coupling part j2, an inner shaft 9 that is inserted into the outer shaft 3 and that is coupled to a distal end of the balloon 5 with a coupling part j1, and a distal end-side marker 7a and a proximal end-side marker 7b disposed on the inner shaft 9, wherein the ends of the distal end-side marker 7a are disposed astride a proximal end coupling position j1p of the coupling part j1 between the balloon 5 and the inner shaft 9 in the longitudinal direction, and therefore, peeling can be prevented at the proximal end coupling position j1p between the balloon 5 and the inner shaft 9, and kinking can be prevented near the end section of the balloon 5 even when the balloon catheter 1 is curved.

Furthermore, because the ends of the proximal end-side marker 7b are disposed astride the proximal end coupling position j2p of the coupling part j2 between the balloon 5 and the outer shaft 3 in the longitudinal direction, peeling can be prevented at the proximal end coupling position j2p between the balloon 5 and the outer shaft 3, and kinking can be prevented near the end section of the balloon 5 even when the balloon catheter 1 is curved.

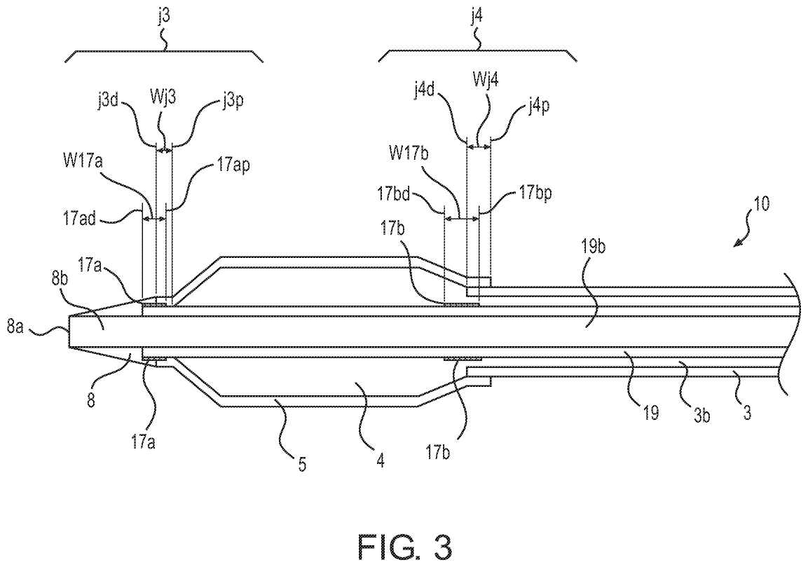

Hereinafter, a balloon catheter of the disclosed embodiments will be described with respect to FIG. 3, but because the overall description of the balloon catheter is the same as that of FIG. 1, the description thereof is omitted, and parts common to the balloon catheter 1 shown in FIGS. 1 and 2 are given the same reference numerals and the description will be omitted.

FIG. 3 is a longitudinal sectional view near the distal end section of a balloon catheter according to the disclosed embodiments.

As shown in FIG. 3, a balloon catheter 10 includes a long outer shaft 3, a balloon 5 provided on the distal end of the outer shaft 3, a distal tip 8 provided on the distal end of the balloon 5, an inner shaft 19 that is inserted into the outer shaft 3 and the balloon 5 and that is coupled to the distal end of the balloon 5, and a connector 2 provided on the proximal end of the outer shaft 3.

The inner shaft 19 of the balloon catheter 10 is made of the same material as the inner shaft 9 of the balloon catheter 1, but it is formed slightly longer than the inner shaft 9 of the balloon catheter 1 because of a relationship with the position in which the distal end-side marker 17a described later is disposed.

That is to say, the inner shaft 19 is also a long and flexible tubular member, is inserted inside the outer shaft 3 and the balloon 5, is coupled to the distal end of the balloon 5, and is provided with a lumen 19b therein, and the proximal end of the inner shaft 19 is coupled to the guide wire port 3a of the outer shaft 3.

Furthermore, the inner shaft 19 is provided with two radiopaque markers, namely a distal end-side marker 17a and a proximal end-side marker 17b, which enable the position of the balloon 5 to be confirmed during a procedure.

Next, the relationship between (i) the coupling position between the balloon 5 and the inner shaft 19, and (ii) the position of the distal end-side marker 17a will be described.

First, as shown in FIG. 3, the coupling position of the coupling part j3 between the balloon 5 and the inner shaft 19 (which corresponds to a “second coupling part”) has a distal end coupling position j3d at the distal end, and a proximal end coupling position j3p at the position of a coupling width Wj3 on the proximal end side from the distal end coupling position j3d.

In contrast, the distal end position 17ad of the distal end-side marker 17a is positioned on the distal end side of the distal end coupling position j3d of the coupling part j3 in the longitudinal direction of the inner shaft 19 (or the balloon catheter 10), and the proximal end position 17ap is at the position of a width W17a on the proximal end side from the distal end position 17ad, and is positioned between the distal end coupling position j3d and the proximal end coupling position j3p of the coupling part j3 in the longitudinal direction.

That is to say, the ends of the distal end-side marker 17a are disposed astride the distal end coupling position j3d of the coupling part j3 between the balloon 5 and the inner shaft 19 in the longitudinal direction.

Therefore, because the ends of the distal end-side marker 17a are disposed astride the distal end coupling position j3d of the coupling part j3 between the balloon 5 and the inner shaft 19 in the longitudinal direction, peeling can be prevented at the distal end coupling position j3d between the balloon 5 and the inner shaft 19, and kinking can be prevented near the end section of the balloon 5 even when the balloon catheter 10 is curved.

Next, the relationship between (i) the coupling position between the balloon 5 and the outer shaft 3, and (ii) the position of the proximal end-side marker 17b will be described.

As shown in FIG. 3, the coupling position of the coupling part j4 between the balloon 5 and the outer shaft 3 (which corresponds to a “first coupling part”) has a distal end coupling position j4d at the distal end, and a proximal end coupling position j4p at the position of a coupling width Wj4 on the proximal end side from the distal end coupling position j4d.

In contrast, the distal end position 17bd of the proximal end-side marker 17b is positioned on the distal end side of the distal end coupling position j4d of the coupling part j4 in the longitudinal direction, and the proximal end position 17bp is at the position of a width W17b on the proximal end side from the distal end position 17bd, and is positioned between the distal end coupling position j4d and the proximal end coupling position j4p of the coupling part j4 in the longitudinal direction.

That is to say, the ends of the proximal end-side marker 17b are disposed astride the distal end coupling position j4d of the coupling part j4 between the balloon 5 and the outer shaft 3 in the longitudinal direction.

Therefore, because the ends of the proximal end-side marker 17b are disposed astride the distal end coupling position j4d of the coupling part j4 between the balloon 5 and the outer shaft 3 in the longitudinal direction, peeling can be prevented at the distal end coupling position j4d between the balloon 5 and the outer shaft 3, and kinking can be prevented near the end section of the balloon 5 even when the balloon catheter 10 is curved.

The same material as the distal end-side marker 7a and the proximal end-side marker 7b of the balloon catheter 1 can be used as the material forming the distal end-side marker 17a and the proximal end-side marker 17b of the balloon catheter 10.

The balloon catheter 10 is provided with a balloon 5, an outer shaft 3 that is coupled to a proximal end of the balloon 5 with a coupling part j4, an inner shaft 19 that is inserted into the outer shaft 3 and that is coupled to a distal end of the balloon 5 with a coupling part j3, and a distal end-side marker 17a and a proximal end-side marker 17b disposed on the inner shaft 19, in which the ends of the distal end-side marker 17a are disposed astride a distal end coupling position j3d of the coupling part j3 between the balloon 5 and the inner shaft 19 in the longitudinal direction, and therefore, peeling can be prevented between at the distal end coupling position j3d between the balloon 5 and the inner shaft 19, and kinking can be prevented near the end section of the balloon 5 even when the balloon catheter 10 is curved.

Furthermore, because the ends of the proximal end-side marker 17b are disposed astride the distal end coupling position j4d of the coupling part j4 between the balloon 5 and the outer shaft 3 in the longitudinal direction, peeling can be prevented at the distal end coupling position j4d between the balloon 5 and the outer shaft 3, and kinking can be prevented near the end section of the balloon 5 even when the balloon catheter 10 is curved.

In the balloon catheter 1, the ends of the distal end-side marker 7a are disposed astride the proximal end coupling position j1p of the coupling part j1 between the balloon 5 and the inner shaft 9 (which corresponds to a “second coupling part”) in the longitudinal direction of the inner shaft 9 (or the balloon catheter 1), and the ends of the proximal end-side marker 7b are disposed astride the proximal end coupling position j2p of the coupling part j2 between the balloon 5 and the outer shaft 3 (which corresponds to a “first coupling part”) in the longitudinal direction.

Furthermore, in the balloon catheter 10, the ends of the distal end-side marker 17a are disposed astride the distal end coupling position j3d of the coupling part j3 between the balloon 5 and the inner shaft 19 (which corresponds to a “second coupling part”) in the longitudinal direction of the inner shaft 19 (or the balloon catheter 10), and the ends of the proximal end-side marker 17b are disposed astride the distal end coupling position j4d of the coupling part j4 between the balloon 5 and the outer shaft 3 (which corresponds to a “first coupling part”) in the longitudinal direction.

However, the present invention is not limited to such configurations, and it is sufficient for the ends of the distal end-side marker to be disposed astride at least one of the distal end coupling position and the proximal end coupling position of the coupling part between the balloon and the inner shaft, and it is sufficient for the ends of the proximal end-side marker to be disposed astride at least one of the distal end coupling position and the proximal end coupling position of the coupling part between the balloon and the outer shaft. In this case, it is possible to prevent peeling of the end section of the coupling part that is disposed between the ends of the marker.

Hereinafter, a balloon catheter of the disclosed embodiments will be described, but because the overall description of the balloon catheter is the same as that in FIG. 1, the description thereof is omitted, and parts common to the balloon catheter 1 shown in FIGS. 1 and 2 are given the same reference numerals and the description will be omitted.

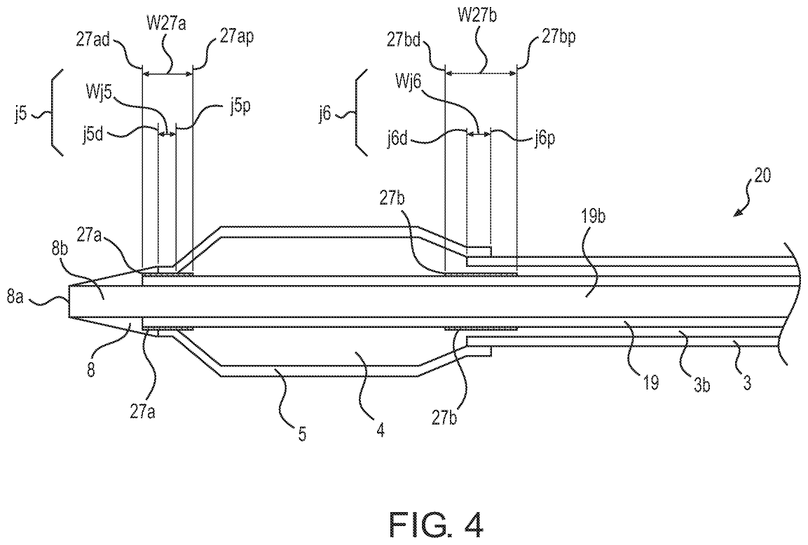

FIG. 4 is a longitudinal sectional view near the distal end section of a balloon catheter according to the disclosed embodiments.

As shown in FIG. 4, the balloon catheter 20 includes a long outer shaft 3, a balloon 5 provided on the distal end of the outer shaft 3, a distal tip 8 provided on the distal end of the balloon 5, an inner shaft 19 that is inserted into the outer shaft 3 and the balloon 5 and that is coupled to the distal end of the balloon 5, and a connector 2 provided on the proximal end of the outer shaft 3.

Furthermore, the inner shaft 19 is provided with two radiopaque markers, namely a distal end-side marker 27a and a proximal end-side marker 27b which enable the position of the balloon 5 to be confirmed during a procedure.

Next, the relationship between (i) the coupling position between the balloon 5 and the inner shaft 19, and (ii) the position of the distal end-side marker 27a will be described.

First, as shown in FIG. 4, the coupling position of the coupling part j5 between the balloon 5 and the inner shaft 19 (which corresponds to a “second coupling part”) has a distal end coupling position j5d at the distal end, and a proximal end coupling position j5p at the position of a coupling width Wj5 on the proximal end side from the distal end coupling position j5d.

In contrast, the distal end position 27ad of the distal end-side marker 27a is positioned on the distal end side of the distal end coupling position j5d of the coupling part j5 in the longitudinal direction of the inner shaft 19 (or the balloon catheter 20), and the proximal end position 27ap is at the position of a width W27a on the proximal end side from the distal end position 27ad, and is positioned on the proximal end side of the proximal end coupling position j5p of the coupling part j5 in the longitudinal direction.

That is to say, the ends of the distal end-side marker 27a are disposed astride both the distal end coupling position j5d and the proximal end coupling position j5p of the coupling part j5 between the balloon 5 and the inner shaft 19 in the longitudinal direction. In other words, the ends of the distal end-side marker 27a are disposed astride the length of the coupling part j5.

Therefore, because the ends of the distal end-side marker 27a are disposed astride both the distal end coupling position j5d and the proximal end coupling position j5p of the coupling part j5 between the balloon 5 and the inner shaft 19 in the longitudinal direction, peeling can be prevented at both the distal end coupling position j5d and the proximal end coupling position j5p between the balloon 5 and the inner shaft 19, and kinking can be further prevented near the end section of the balloon 5 even when the balloon catheter 20 is curved.

Next, the relationship between (i) the coupling position between the balloon 5 and the outer shaft 3, and (ii) the position of the proximal end-side marker 27b will be described.

As shown in FIG. 4, the coupling position of the coupling part j6 between the balloon 5 and the outer shaft 3 (which corresponds to the “first coupling part”) has a distal end coupling position j6d at the distal end, and a proximal end coupling position j6p at the position of a coupling width Wj6 on the proximal end side from the distal end coupling position j6d.

In contrast, the distal end position 27bd of the proximal end-side marker 27b is positioned on the distal end side of the distal end coupling position j6d of the coupling part j6 in the longitudinal direction, and the proximal end position 27bp is at the position of the width W27b on the proximal end side from the distal end position 27bd, and is positioned on the proximal end side of the proximal end coupling position j6p of the coupling part j6 in the longitudinal direction.

That is to say, the ends of the proximal end-side marker 27b are disposed astride both the distal end coupling position j6d and the proximal end coupling position j6p of the coupling part j6 between the balloon 5 and the outer shaft 3 in the longitudinal direction.

Therefore, because the ends of the proximal end-side marker 27b are disposed astride both the distal end coupling position j6d and the proximal end coupling position j6p of the coupling part j6 between the balloon 5 and the outer shaft 3 in the longitudinal direction, peeling can be prevented at both the distal end coupling position j6d and the proximal end coupling position j6p of the coupling part j6 between the balloon 5 and the outer shaft 3, and kinking can be prevented near the end section of the balloon 5 even when the balloon catheter 20 is curved.

The same material as the distal end-side marker 7a and the proximal end-side marker 7b of the balloon catheter 1 can be used as the material forming the distal end-side marker 27a and the proximal end-side marker 27b of the balloon catheter 20.

The balloon catheter 20 is provided with a balloon 5, an outer shaft 3 that is coupled to a proximal end of the balloon 5 with a coupling part j6, an inner shaft 19 that is inserted into the outer shaft 3 and that is coupled to a distal end of the balloon 5 with a coupling part j5, and a distal end-side marker 27a and a proximal end-side marker 27b disposed on the inner shaft 19, in which the ends of the distal end-side marker 27a are disposed astride both a distal end coupling position j5d and a proximal end coupling position j5p of the coupling part j5 in the longitudinal direction, and the ends of the proximal end-side marker 27b are disposed astride both a distal end coupling position j6d and a proximal end coupling position j6p of the coupling part j6 in the longitudinal direction, and therefore, peeling can be further prevented at both the distal end coupling position j5d and the proximal end coupling position j5p of the coupling part j5 between the balloon 5 and the inner shaft 19, and at both the distal end coupling position j6d and the proximal end coupling position j6p of the coupling part j6 between the balloon 5 and the outer shaft 3, and kinking can be prevented near the end section of the balloon even when the balloon catheter 20 is curved.

In the balloon catheter 20, the ends of the distal end-side marker 27a are disposed astride both the distal end coupling position j5d and the proximal end coupling position j5p of the coupling part j5 between the balloon 5 and the inner shaft 19 in the longitudinal direction, and the ends of the proximal end-side marker 27b are disposed astride both the distal end coupling position j6d and the proximal end coupling position j6p of the coupling part j6 between the balloon 5 and the outer shaft 3 in the longitudinal direction.

However, the present invention is not limited to such configurations, and as long as at least the ends of the distal end-side marker 27a or the ends of the proximal end-side marker 27b are disposed astride both the distal end coupling position and the proximal end coupling position of the corresponding coupling part, peeling can be prevented at the distal end coupling position and the proximal end coupling position of that coupling part, and kinking of the balloon can be prevented near that coupling part.

Hereinafter, a balloon catheter of the disclosed embodiments will be described, but because the overall description of the balloon catheter is the same as that in FIG. 1, the description thereof is omitted, and parts common to the balloon catheter 1 shown in FIGS. 1 and 2 are given the same reference numerals and the description will be omitted.

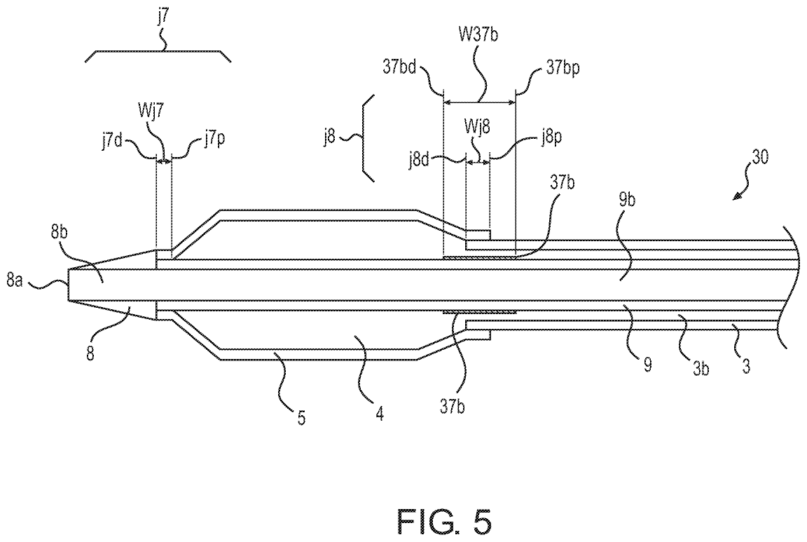

FIG. 5 is a longitudinal sectional view near the distal end section of a balloon catheter according to the disclosed embodiments.

As shown in FIG. 5, the balloon catheter 30 includes a long outer shaft 3, a balloon 5 provided on the distal end of the outer shaft 3, a distal tip 8 provided on the distal end of the balloon 5, an inner shaft 9 that is inserted into the outer shaft 3 and the balloon 5 and that is coupled to the distal end of the balloon 5, and a connector 2 provided on the proximal end of the outer shaft 3.

Furthermore, the inner shaft 9 is provided with a radiopaque proximal end-side marker 37b which enables the position of the balloon 5 to be confirmed during a procedure.

Furthermore, the inner shaft 9 is not provided with a radiopaque marker on the distal end side of the balloon 5. However, as shown in FIG. 5, the coupling position of the coupling part j7 between the balloon 5 and the inner shaft 9 has a distal end coupling position j7d at the distal end, and a proximal end coupling position j7p at the position of a coupling width Wj7 on the proximal end side from the distal end coupling position j7d.

Here, the relationship between (i) the coupling position between the balloon 5 and the outer shaft 3, and (ii) the position of the proximal end-side marker 37b will be described.

As shown in FIG. 5, the coupling position of the coupling part j8 between the balloon 5 and the outer shaft 3 (which corresponds to a “first coupling part”) has a distal end coupling position j8d at the distal end, and a proximal end coupling position j8p at the position of a coupling width Wj8 on the proximal end side from the distal end coupling position j8d.

In contrast, the distal end position 37bd of the proximal end-side marker 37b is positioned on the distal end side of the distal end coupling position j8d of the coupling part j8 in the longitudinal direction of the inner shaft 9 (or the balloon catheter 30), and the proximal end position 37bp is at the position of a width W37b on the proximal end side from the distal end position 37bd, and is positioned on the proximal end side of the proximal end coupling position j8p of the coupling part j8 in the longitudinal direction.

That is to say, the ends of the proximal end-side marker 37b are disposed astride both the distal end coupling position j8d and the proximal end coupling position j8p of the coupling part j8 between the balloon 5 and the outer shaft 3 in the longitudinal direction.

Therefore, because the ends of the proximal end-side marker 37b are disposed astride both the distal end coupling position j8d and the proximal end coupling position j8p of the coupling part j8 between the balloon 5 and the outer shaft 3 in the longitudinal direction, peeling can be prevented at both the distal end coupling position j8d and the proximal end coupling position j8p of the coupling part j8 between the balloon 5 and the outer shaft 3, and kinking can be prevented near the end section of the balloon 5 even when the balloon catheter 30 is curved.

The same material as the distal end-side marker 7a and the proximal end-side marker 7b of the balloon catheter 1 can be used as the material forming the proximal end-side marker 37b.

The balloon catheter 30 is provided with a balloon 5, an outer shaft 3 that is coupled to a proximal end of the balloon 5 with a coupling part j8, an inner shaft 9 that is inserted into the outer shaft 3 and that is coupled to a distal end of the balloon 5 with a coupling part j7, and a proximal end-side marker 37b disposed on the inner shaft 9, in which a marker is not disposed on the distal end-side coupling part j7 and is disposed on the proximal end-side coupling part j8, and therefore, the distal end section of the balloon catheter 30 can be made even thinner and softer.

Furthermore, because the ends of the distal end-side marker 37b are disposed astride both the distal end coupling position j8d and the proximal end coupling position j8p of the proximal end-side coupling part j8 in the longitudinal direction, peeling can be prevented at the distal end coupling position j8d and the proximal end coupling position j8p between the balloon 5 and the outer shaft 3, and kinking can be prevented near the end section of the balloon 5 even when the balloon catheter 30 is curved.

Hereinafter, a balloon catheter of the disclosed embodiments will be described, but because the overall description of the balloon catheter is the same as that in FIG. 1, the description thereof is omitted, and parts common to the balloon catheter 1 shown in FIGS. 1 and 2 are given the same reference numerals and the description will be omitted.

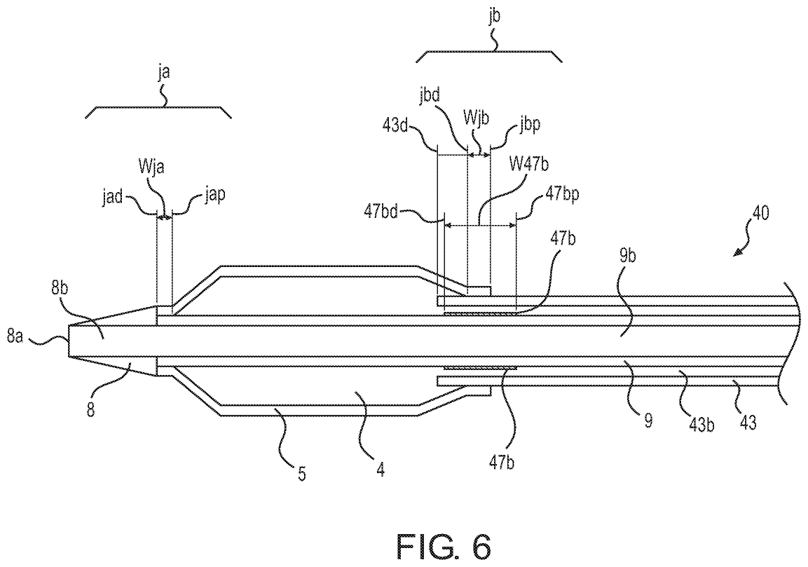

FIG. 6 is a longitudinal sectional view near the distal end section of a balloon catheter according to the disclosed embodiments.

As shown in FIG. 6, the balloon catheter 40 includes a long outer shaft 43, a balloon 5 provided on the distal end section of the outer shaft 43, a distal tip 8 provided on the distal end of the balloon 5, an inner shaft 9 that is inserted into the outer shaft 43 and the balloon 5 and that is coupled to the distal end of the balloon 5, and a connector 2 provided on the proximal end of the outer shaft 3.

The outer shaft 43 is a long and flexible tubular member, and as shown in FIG. 6, the distal end 43d extends beyond the distal end of the coupling part jb between the proximal end of the balloon 5 and the outer shaft 43 on the distal end side, and the proximal end is coupled to the distal end of the connector 2.

The outer shaft 43 has an inner shaft 9 inserted therein, and has a lumen 43b between the outer shaft and the inner shaft 9 through which the internal space 4 of the balloon 5 communicates with a lumen inside the connector 2 (not illustrated).

Furthermore, along the longitudinal direction, the outer shaft 43 is provided with, in a similar fashion to the outer shaft 3, a guide wire port 43a (not illustrated) which serves as an opening for inserting a guide wire, and the guide wire port 43a is coupled to the proximal end of the inner shaft 9.

The same material as the material forming the outer shaft 3 can be used to form the outer shaft 43.

The inner shaft 9 of the balloon catheter 40 is provided with a radiopaque proximal end-side marker 47b which enables the position of the balloon 5 to be confirmed during a procedure.

Furthermore, the inner shaft 9 is not provided with a radiopaque marker on the distal end side of the balloon 5. However, as shown in FIG. 6, the coupling position of the coupling part ja between the balloon 5 and the inner shaft 9 has a distal end coupling position jad at the distal end, and a proximal end coupling position jap at the position of a coupling width Wja on the proximal end side from the distal end coupling position jad.

Here, the relationship between (i) the coupling position between the balloon 5 and the outer shaft 43, and (ii) the position of the proximal end-side marker 47b will be described.

As shown in FIG. 6, the coupling position of the coupling part jb between the balloon 5 and the outer shaft 43 (which corresponds to a “first coupling part”) has a distal end coupling position jbd at the distal end, and a proximal end coupling position jbp at the position of a coupling width Wjb on the proximal end side from the distal end coupling position jbd.

In contrast, the distal end position 47bd of the proximal end-side marker 47b is positioned on the distal end side of the distal end coupling position jbd of the coupling part jb in the longitudinal direction of the inner shaft 9 (or the balloon catheter 40), and the proximal end position 47bp is at the position of a width W47b on the proximal end side from the distal end position 47bd, and is positioned on the proximal end side of the proximal end coupling position jbp of the coupling part jb in the longitudinal direction.

That is to say, the ends of the proximal end-side marker 47b are disposed astride both the distal end coupling position jbd and the proximal end coupling position jbp of the coupling part jb between the balloon 5 and the outer shaft 43 in the longitudinal direction.

Further, as mentioned above, the distal end 43d of the outer shaft 43 extends beyond the distal end jbd of the coupling part jb on the distal end side.

Therefore, because the ends of the proximal end-side marker 47b are disposed astride both the distal end coupling position jbd and the proximal end coupling position jbp of the coupling part jb between the balloon 5 and the outer shaft 43 in the longitudinal direction, and the distal end 43d of the outer shaft 43 extends beyond the distal end coupling position jbd of the coupling part jb on the distal end side, the change in rigidity near the distal end section of the balloon 5 is made more gradual in the longitudinal direction, and therefore, peeling can be further prevented at the distal end coupling position jbd and the proximal end coupling position jbp between the balloon 5 and the outer shaft 43, and kinking can be further prevented near the end section of the balloon 5 even when the balloon catheter 40 is curved.

The same material as the distal end-side marker 7a and the proximal end-side marker 7b of the balloon catheter 1 can be used as the material forming the proximal end-side marker 47b.

The balloon catheter 40 is provided with a balloon 5, an outer shaft 43 that is coupled to a proximal end of the balloon 5 with a coupling part jb, an inner shaft 9 that is inserted into the outer shaft 43 and that is coupled to a distal end of the balloon 5 with a coupling part ja, and a proximal end-side marker 37b disposed on the inner shaft 9, in which a marker is not disposed on the coupling part ja and is disposed on the coupling part jb, and therefore, the distal end section of the balloon catheter 40 can be made even thinner and softer.

Furthermore, because the ends of the proximal end-side marker 47b are disposed astride both the distal end coupling position jbd and the proximal end coupling position jbp of the coupling part jb between the balloon 5 and the outer shaft 43 in the longitudinal direction, and the distal end 43d of the outer shaft 43 extends beyond the distal end jbd of the coupling part jb on the distal end side, the change in rigidity near the distal end section of the balloon 5 is made more gradual in the longitudinal direction, and therefore, peeling can be further prevented at the distal end coupling position jbd and the proximal end coupling position jbp between the balloon 5 and the outer shaft 43, and kinking can be further prevented near the end section of the balloon 5 even when the balloon catheter 40 is curved.

Hereinafter, a balloon catheter of the disclosed embodiments will be described, but because the overall description of the balloon catheter is the same as that in FIG. 1, the description thereof is omitted, and parts common to the balloon catheter 1 shown in FIGS. 1 and 2 are given the same reference numerals and the description will be omitted.

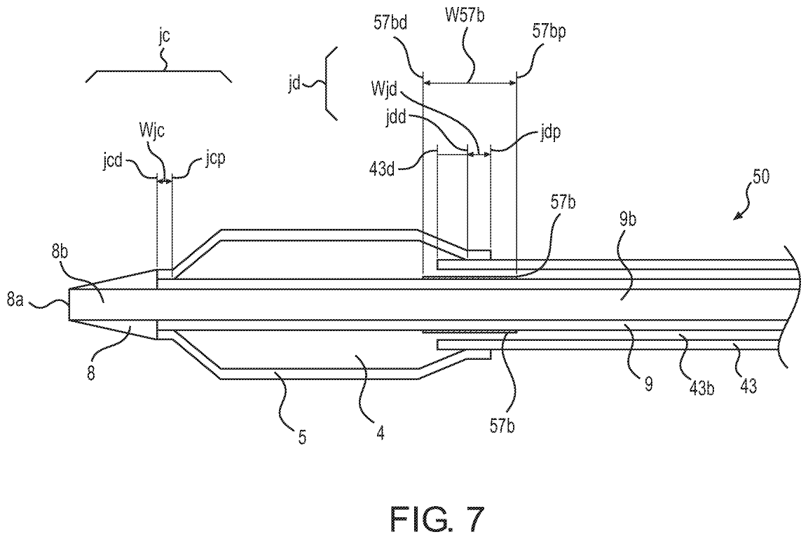

FIG. 7 is a longitudinal sectional view near the distal end section of a balloon catheter according to the disclosed embodiments.

As shown in FIG. 7, the balloon catheter 50 includes a long outer shaft 43, a balloon 5 provided on the distal end of the outer shaft 43, a distal tip 8 provided on the distal end of the balloon 5, an inner shaft 9 that is inserted into the outer shaft 43 and the balloon 5 and that is coupled to the distal end of the balloon 5, and a connector 2 provided on the proximal end of the outer shaft 3.

The inner shaft 9 of the balloon catheter 50 is provided with a radiopaque proximal end-side marker 57b, which enables the position of the balloon 5 to be confirmed during a procedure.

Furthermore, the inner shaft 9 is not provided with a radiopaque marker on the distal end side of the balloon 5. However, as shown in FIG. 7, the coupling position of the coupling part jc between the balloon 5 and the inner shaft 9 has a distal end coupling position jcd at the distal end, and a proximal end coupling position jcp at the position of a coupling width Wjc on the proximal end side from the distal end coupling position jcd.

Here, the relationship between (i) the coupling position between the balloon 5 and the outer shaft 43, and (ii) the position of the proximal end-side marker 57b will be described.

As shown in FIG. 7, the coupling position of the coupling part jd between the balloon 5 and the outer shaft 43 (which corresponds to a “first coupling part”) has a distal end coupling position jdd at the distal end, and a proximal end coupling position jdp at the position of a coupling width Wjd on the proximal end side from the distal end coupling position jdd.

In contrast, the distal end position 57bd of the proximal end-side marker 57b is positioned on the distal end side of the distal end coupling position jdd of the coupling part jd in the longitudinal direction of the inner shaft 9 (or the balloon catheter 50), and is positioned on the distal end side of the distal end 43d of the outer shaft 43 in the longitudinal direction.

Furthermore, the proximal end position 57bp is at the position of a width W57b on the proximal end side from the distal end position 57bd, and is positioned on the proximal end side of the proximal end coupling position jdp of the coupling part jd in the longitudinal direction.

That is to say, the ends of the proximal end-side marker 57b are disposed astride both the distal end coupling position jdd and the proximal end coupling position jdp of the coupling part jd between the balloon 5 and the outer shaft 43 in the longitudinal direction.

Further, as mentioned above, the distal end position 57bd of the proximal end-side marker 57b extends beyond the distal end 43d of the outer shaft 43 on the distal end side in the longitudinal direction.

Therefore, because the ends of the proximal end-side marker 57b are disposed astride both the distal end coupling position jdd and the proximal end coupling position jdp of the coupling part jd between the balloon 5 and the outer shaft 43 in the longitudinal direction, the distal end of the outer shaft 43 extends beyond the distal end jdd of the coupling part jd on the distal end side, and the distal end position 57bd of the proximal end-side marker 57b extends beyond the distal end 43d of the outer shaft 43 on the distal end side, peeling can be further prevented at the distal end coupling position jdd and the proximal end coupling position jdp between the balloon 5 and the outer shaft 43, and kinking can be prevented near the end section of the balloon 5 even when the balloon catheter 50 is curved, and further, the balloon can be deflated more safely by preventing the necking phenomenon from occurring at the proximal end section of the balloon 5.

The same material as the distal end-side marker 7a and the proximal end-side marker 7b of the balloon catheter 1 can be used as the material forming the proximal end-side marker 57b.

The balloon catheter 50 is provided with a balloon 5, an outer shaft 43 that is coupled to a proximal end of the balloon 5 with a coupling part jd, an inner shaft 9 that is inserted into the outer shaft 43 and that is coupled to a distal end of the balloon 5 with a coupling part jc, and a proximal end-side marker 57b disposed on the inner shaft 9, in which a marker is not disposed on the coupling part jc but is disposed on the coupling part jd, and therefore, the distal end section of the balloon catheter 50 can be made even thinner and softer.

Furthermore, because the ends of the proximal end-side marker 57b are disposed astride both the distal end coupling position jdd and the proximal end coupling position jdp of the coupling part jd between the balloon 5 and the outer shaft 43 in the longitudinal direction, the distal end of the outer shaft 43 extends beyond the distal end coupling position jdd of the coupling part jd on the distal end side, and further, the distal end position 57bd of the proximal end-side marker 57b extends beyond the distal end 43d of the outer shaft 43 on the distal end side, peeling can be further prevented at the distal end coupling position jdd and the proximal end coupling position jdp between the balloon 5 and the outer shaft 43, and kinking can be prevented near the end section of the balloon 5 even when the balloon catheter 50 is curved, and further, the balloon can be deflated more safely by preventing the necking phenomenon from occurring at the proximal end section of the balloon 5.

The balloon catheter according to various embodiments of the present invention has been described above, however, the present invention is not limited to the above embodiments, and various modifications can be made without departing from the scope of the invention.

For example, the balloon catheters 30, 40, and 50 were described with no radiopaque marker being provided at a position on the inner shaft 9 corresponding to the distal end side of the balloon 5; however, if the distal end section of the balloon catheter does not need to be made thinner, or the distal end section of the catheter does not need to be made soft, a radiopaque marker can be provided at a position corresponding to the distal end side of the balloon 5 in the same manner as the balloon catheter 1 and the balloon catheter 10.

1. A balloon catheter comprising:
a balloon;
an outer shaft that is coupled to a proximal end of the balloon with a first coupling part;
an inner shaft that disposed within the outer shaft and that is coupled to a distal end of the balloon with a second coupling part; and
a first marker disposed on the inner shaft at the first coupling part,
wherein at least one of a distal end and a proximal end of the first coupling part is disposed between a distal end and a proximal end of the first marker, in a longitudinal direction of the inner shaft.

2. The balloon catheter according to claim 1, wherein both the distal end and the proximal end of the first coupling part are disposed between the distal end and the proximal end of the first marker, in the longitudinal direction.

3. The balloon catheter according to claim 1, wherein the distal end of the outer shaft extends distally beyond the distal end of the first coupling part.

4. The balloon catheter according to claim 3, wherein the distal end of the first marker extends distally beyond the distal end of the outer shaft.

5. The balloon catheter according to claim 1, wherein there is no marker disposed at the second coupling part.

6. The balloon catheter according to claim 2, wherein there is no marker disposed at the second coupling part.

7. The balloon catheter according to claim 3, wherein there is no marker disposed at the second coupling part.

8. The balloon catheter according to claim 4, wherein there is no marker disposed at the second coupling part.

9. The balloon catheter according to claim 1, further comprising:
a second marker disposed on the inner shaft at the second coupling part,
wherein at least one of a distal end and a proximal end of the second coupling part is disposed between a distal end and a proximal end of the second marker, in the longitudinal direction.

10. The balloon catheter according to claim 9, wherein both the distal end and the proximal end of the second coupling part are disposed between the distal end and the proximal end of the second marker, in the longitudinal direction.
